(19) United States
(12) Patent Application Publication
(10) Pub. No.: US 2023/0337938 A1
(43) Pub. Date: Oct. 26, 2023

(54) METHOD AND APPARATUS TO ENSURE BIOSAFETY BY FILTERING EXHALED BREATH WASTE DURING BREATH SAMPLING

(71) Applicant: Breathonix Pte. Ltd., Singapore (SG)

(72) Inventors: Zhunan JIA, Singapore (SG); Fang Du, Singapore (SG); Wei Qiang Ong, Singapore (SG); Adhiraj Mahajan, Singapore (SG)

(73) Assignee: Breathonix Pte. Ltd., Singapore (SG)

(21) Appl. No.: 17/727,193

(22) Filed: Apr. 22, 2022

(51) Int. Cl. A61B 5/097; A61B 5/08

(52) U.S. Cl. CPC A61B 5/097; A61B 5/082

(57) ABSTRACT

An apparatus (100) for analysing a breath sample includes a breath sampler (11), an expandable breath reservoir (20) fluidly connected to the breath sampler (11), a pump (30) fluidly connected to the expandable breath reservoir (20), and a breath analyser (40) fluidly connected to the breath sampler (11). The expandable breath reservoir (20) is configured to expand when a subject breathes into the breath sampler (11) and the pump (30) is configured to remove the contents of the expandable breath reservoir (20) after expansion.

### FIELD

The present disclosure relates to the field of breath analysis and virology. In particular, the disclosure relates to an apparatus, and teaches a method of ensuring biosafety by preventing the escape of respiratory viral infection particulates (e.g. by a coronavirus or influenza virus) in the breath of a subject during breath sampling.

### BACKGROUND

Respiratory viral infections are viral infections that affect the respiratory tract. Common viruses that cases respiratory viral infections include coronavirus, influenza virus, respiratory syncytial virus (RSV) and rhinovirus. Many of these viruses are seasonal in prevalence and can be affected by environmental conditions such as humidity and temperature. From time to time, there are novel strains of viruses that lead to a widespread pandemic.

There is a need for effective methods to rapidly determine whether a subject is suffering from respiratory viral infection, such as during a pandemic. A new method for rapid screening of respiratory viral infection is the use of a breath test to analyse the volatile organic compounds in exhaled breath. However, despite the many advantages of using breath tests of for screening respiratory viral infection, such as being non-invasive, simple, and fast, there remain a biosafety concern of respiratory viral infection particulates in the exhaled breath being exhaust into the environment.

### SUMMARY OF THE INVENTION

Disclosed herein is a method of ensuring biosafety during breath sampling where the respiratory viral infection particulates in the exhaled waste breath (i.e. the portion of the breath which is not analysed) are being filtered before being released into the environment, while at the same time, not affecting the performance and accuracy of the analysis of the breath.

The invention provides an apparatus (100) for analysing a breath sample, the apparatus comprising:
a breath sampler (11);
an expandable breath reservoir (20) fluidly connected to the breath sampler (11);
a pump (30) fluidly connected to the expandable breath reservoir (20); and
a breath analyser (40) fluidly connected to the breath sampler (11), wherein:
the expandable breath reservoir (20) is configured to expand when a subject breathes into the breath sampler (11); and
the pump (30) is configured to remove the contents of the expandable breath reservoir (20) after expansion.

The parts of the apparatus form a fluid flow path from the breath sampler to the breath analyser, and also from the breath sampler to the expandable breath reservoir and then to the pump. Breath may be removed from the apparatus by the pump, preferably via a filter configured to remove viral particles (e.g. a HEPA filter). In this way, the contents of an exhaled breath may be analysed by the breath analyser (e.g. a mass spectrometer) and then efficiently removed from the apparatus without danger of infection for nearby persons.

The invention also provides a method for taking a breath sample from a patient using an apparatus according to the invention, the method comprising:
(i) the patient blowing air into the breath sampler (11), thereby providing breath into the expandable breath reservoir (20) and breath analyser (40); and
(ii) removing waste breath from the expandable breath reservoir (20) by activating the pump (30).

### BRIEF DESCRIPTION OF THE DRAWINGS

FIG. 1 depicts a schematic of the apparatus 100 and its components of the method to ensure biosafety during breath sampling.

FIG. 2 illustrates a side view of the apparatus 100 of the present invention to ensure biosafety during breath sampling.

### DETAILED DESCRIPTION OF THE INVENTION

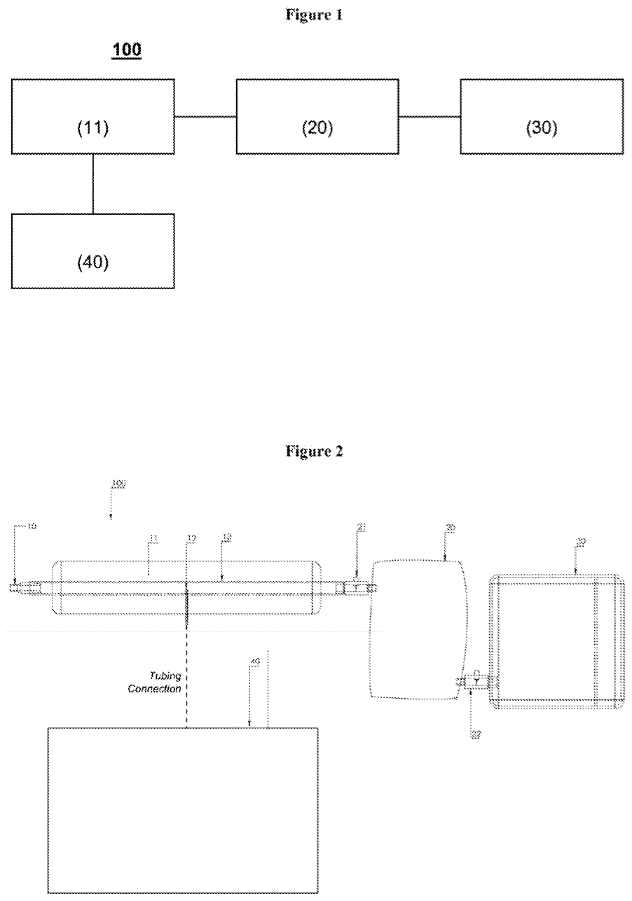

FIG. 1 shows an embodiment of the present invention consisting of the breath sampler 11, breath reservoir 20, suction pump 30 and a mass spectrometer 40. The breath sampler 11 is connected to both the breath reservoir 20 and mass spectrometer 40. The breath reservoir 20 is in turn connected to the suction pump 30.

FIG. 2 shows a detailed illustration of particular embodiment of the present invention. During the sampling process, a subject will breathe into the system through mouthpiece 10, which is inserted into a sampling tube 13 in the breath sampler 11. A portion of the breath will enter the mass spectrometer 40 through a metal needle 12. The remaining waste breath will enter the expandable breath reservoir 20 via a connector valve 21. The other end of the breath reservoir 20 is connected to a suction pump 30 via another connector valve 22.

The terms “subject”, “patient”, “host” or “individual” used interchangeably herein, refer to any subject. The term “subject” includes any human or non-human animal. In one embodiment, the subject is a human. The term “non-human animal” includes all vertebrates. e.g., mammals and non-mammals, such as non-human primates, sheep, dog, cow, chickens, amphibians, reptiles, etc.

The terms “particulate”, “particle”, “aerosol particle”, “droplet”, “airborne particle” or “particulate matter” used interchangeably herein, refer to any particulate. The term “particulate” includes any solid or liquid particulate of any size.

The term “respiratory viral infection” as used herein refers to an infection due to a respiratory virus. The term “respiratory virus” refers to a virus which infects cells of the respiratory tract, such as cells lining the oral cavity, nasopharynx, throat, larynx, bronchi and bronchioles, etc. Respiratory viruses include influenza virus, rhinovirus, adenovirus, coronavirus, respiratory syncytial virus (RSV), measles virus, mumps virus, parainfluenza virus, rubella virus, poxvirus, parvovirus, hantavirus and varicella virus.

“Coronavirus” as used herein refers to a genus in the family Coronaviridae, which family is in turn classified within the order Nidovirales. The coronaviruses are large, enveloped, positive-stranded RNA viruses. They have the largest genomes of all RNA viruses and replicate by a unique mechanism that results in a high frequency of recombination. The coronaviruses include alphacoronavirus, betacoronavirus, gammacoronavirus and deltacoronavirus. Non-limiting examples of coronaviruses include SARS coronavirus (SARS-CoV or SARS-CoV-2). MERS coronavirus, transmissible gastroenteritis virus (TGEV), human respiratory coronavirus, porcine respiratory coronavirus, canine coronavirus, feline enteric coronavirus, feline infectious peritonitis virus, rabbit coronavirus, murine hepatitis virus, sialodacryoadenitis virus, porcine hemagglutinating encephalomyelitis virus, bovine coronavirus, avian infectious bronchitis virus, and turkey coronavirus, as well as chimeras of any of the foregoing.

In one embodiment, the respiratory viral infection is a coronavirus infection. The coronavirus infection may be a COVID-19 infection (or an infection by SARS-CoV-2 virus).

The method as defined herein may comprise filtering the likelihood of a respiratory viral infection from the subject. The term “likelihood of a respiratory viral infection” may refer to how likely it is for a respiratory viral infection to be present in a subject.

The method as defined herein may comprise filtering the likelihood of a respiratory viral infection particulate from the subject. The term “likelihood of a respiratory viral infection particulate” may refer to how likely it is for a respiratory viral particulate to be present in a subject's breath.

The expandable breath reservoir (20) may have a volume of 0.5 litres, 0.6 litres, 0.7 litres, 0.8 litres, 0.9 litres, 1.0 litres, 2.0 litres, 3.0 litres, 4.0 litres, 5.0 litres, 6.0 litres, 7.0 litres, 8.0 litres, 9.0 litres or 10.0 litres or anywhere in between. In other words, the expandable breath reservoir (20) may have any of the following volumes:
0.5-0.6 litres, 0.5-0.7 litres, 0.5-0.8 litres, 0.5-0.9 litres, 0.5-1.0 litres, 0.5-2.0 litres, 0.5-3.0 litres, 0.5-4.0 litres, 0.5-5.0 litres, 0.5-6.0 litres, 0.5-7.0 litres, 0.5-8.0 litres, 0.5-9.0 litres, 0.5-10.0 litres;
0.6-0.7 litres, 0.6-0.8 litres, 0.6-0.9 litres, 0.6-1.0 litres, 0.6-2.0 litres, 0.6-3.0 litres, 0.6-4.0 litres, 0.6-5.0 litres, 0.6-6.0 litres, 0.6-7.0 litres, 0.6-8.0 litres, 0.6-9.0 litres, 0.6-10.0 litres;
0.7-0.8 litres, 0.7-0.9 litres, 0.7-1.0 litres, 0.7-2.0 litres, 0.7-3.0 litres, 0.7-4.0 litres, 0.7-5.0 litres, 0.7-6.0 litres, 0.7-7.0 litres, 0.7-8.0 litres, 0.7-9.0 litres, 0.7-10.0 litres;
0.8-0.9 litres, 0.8-1.0 litres, 0.8-2.0 litres, 0.8-3.0 litres, 0.8-4.0 litres, 0.8-5.0 litres, 0.8-6.0 litres, 0.8-7.0 litres, 0.8-8.0 litres, 0.8-9.0 litres, 0.8-10.0 litres;
0.9-1.0 litres, 0.9-2.0 litres, 0.9-3.0 litres, 0.9-4.0 litres, 0.9-5.0 litres, 0.9-6.0 litres, 0.9-7.0 litres, 0.9-8.0 litres, 0.9-9.0 litres, 0.9-10.0 litres;
1.0-2.0 litres, 1.0-3.0 litres, 1.0-4.0 litres, 1.0-5.0 litres, 1.0-6.0 litres, 1.0-7.0 litres, 1.0-8.0 litres, 1.0-9.0 litres, 1.0-10.0 litres;
2.0-3.0 litres, 2.0-4.0 litres, 2.0-5.0 litres, 2.0-6.0 litres, 2.0-7.0 litres, 2.0-8.0 litres, 2.0-9.0 litres, 2.0-10.0 litres;
3.0-4.0 litres, 3.0-5.0 litres, 3.0-6.0 litres, 3.0-7.0 litres, 3.0-8.0 litres, 3.0-9.0 litres, 3.0-10.0 litres;
4.0-5.0 litres, 4.0-6.0 litres, 4.0-7.0 litres, 4.0-8.0 litres, 4.0-9.0 litres, 4.0-10.0 litres;
5.0-6.0 litres, 5.0-7.0 litres, 5.0-8.0 litres, 5.0-9.0 litres, 5.0-10.0 litres;
6.0-7.0 litres, 6.0-8.0 litres, 6.0-9.0 litres, 6.0-10.0 litres;
7.0-8.0 litres, 7.0-9.0 litres, 7.0-10.0 litres;
8.0-9.0 litres, 8.0-10.0 litres; and
9.0-10.0 litres.

Disclosed herein are embodiments of the invention according to the following numbered clauses.
1. An apparatus (100) for ensuring biosafety during breath sampling, the apparatus comprising:
   a mouthpiece (10);
   a breath sampler (11);
   an expandable breath reservoir (20);
   at least 2 connector values (21, 22);
   a suction pump (30), and
   a mass spectrometer (40)
2. The apparatus (100) of clause 1, comprises a mouthpiece (10) which contains a one-way valve for one-way flow of breath; the breath can only flow into the breath reservoir (20) and mass spectrometer (40) and not out of the apparatus via the mouthpiece (10).
3. The apparatus (100) of clause 1, comprises a mouthpiece (10) which is disposable.
4. The apparatus (100) of clause 1, comprises an expandable breath reservoir (20) which is disposable.
5. The apparatus (100) of clause 1, comprises an expandable breath reservoir (20) which can be of volume of 0.5 litres, 0.6 litres, 0.7 litres, 0.8 litres, 0.9 litres, 1.0 litres, 2.0 litres, 3.0 litres, 4.0 litres, 5.0 litres, 6.0 litres, 7.0 litres, 8.0 litres, 9.0 litres or 10.0 litres or anywhere in between.
6. The apparatus (100) of clause 1, comprises of at least two connector valves (21, 22) that can be activated either mechanically or electrically.
7. The apparatus (100) of clause 1, comprises of at least two connector valves (21, 22) that can be in an ‘open’ or ‘close’ state independently.
8. The apparatus (100) of clause 1, comprising a suction pump (30) that can contain at least one layer of HEPA filter in the pump to filter respiratory viral infection particulates.
9. The apparatus (100) of clause 1, comprising a suction pump (30) that can be connected to a device containing at least one layer of HEPA filter to filter respiratory viral infection particulates.
10. A method for ensuring biosafety during breath sampling using an apparatus (100) comprising at least one mouthpiece (10); a breath sampler (11); an expandable breath reservoir (20); at least two connector values (21, 22), a suction pump (30) and a mass spectrometer (40); the method comprising:
blowing (1) air into breath reservoir (20) through mouthpiece (10), breath sampler (11) and a connector value (21) that is ‘opened’ during breath sampling. Connector value (22) is ‘closed’ in this step.
exhausting (2) waste breath from breath reservoir (20) by opening connector valve (22) filtering (3) respiratory viral infection particulate through HEPA filter in suction pump (30) or a device containing HEPA filter that is attached to suction pump (30).

1. An apparatus (100) for analysing a breath sample, the apparatus comprising:
a breath sampler (11);
an expandable breath reservoir (20) fluidly connected to the breath sampler (11);
a pump (30) fluidly connected to the expandable breath reservoir (20); and
a breath analyser (40) fluidly connected to the breath sampler (11),
wherein:
the expandable breath reservoir (20) is configured to expand when a subject breathes into the breath sampler (11); and
the pump (30) is configured to remove the contents of the expandable breath reservoir (20) after expansion.

2. The apparatus (100) according to claim 1, wherein the breath sampler (11) comprises a mouthpiece (10),
optionally wherein the mouthpiece (10) is disposable.

3. The apparatus (100) according to claim 2, wherein the mouthpiece (10) comprises a one-way valve configured to prevent flow of breath out of the apparatus via the mouthpiece (10).

4. The apparatus (100) according to claim 1, wherein the pump (30) is a suction pump.

5. The apparatus (100) according to claim 1 wherein the breath analyser (40) is a mass spectrometer.

6. The apparatus (100) according to claim 1, further comprising:
a first connector valve (21) located between the breath sampler (11) and expandable breath reservoir (20); and/or
a second connector valve (22) located between the expandable breath reservoir (20) and pump (30).

7. The apparatus (100) according to claim 6, wherein the first connector valve (21) and/or the second connector valve (22) are mechanically or electrically activatable.

8. The apparatus (100) according to claim 6, wherein the first connector valve (21) and/or the second connector valve (22) are independently openable.

9. The apparatus (100) according to claim 1, wherein the pump (30) comprises a filter configured to remove viral particles.

10. The apparatus (100) according to claim 1, wherein the pump (30) is fluidly connected to a device comprising a filter configured to remove viral particles.

11. The apparatus (100) according to claim 9, wherein the filter configured to remove viral particles is a HEPA filter.

12. The apparatus according to claim 1, wherein the expandable breath reservoir (20) is disposable.

13. The apparatus according to claim 1, wherein the expandable breath reservoir (20) has a volume of from 0.5 to 10 litres.

14. A method for taking a breath sample from a patient using an apparatus according to claim 1, the method comprising:
(i) the patient blowing air into the breath sampler (11), thereby providing breath into the expandable breath reservoir (20) and breath analyser (40); and
(ii) removing waste breath from the expandable breath reservoir (20) by activating the pump (30).

15. The method according to claim 14, wherein the apparatus comprises:
a first connector valve (21) located between the breath sampler (11) and expandable breath reservoir (20); and
a second connector valve (22) located between the expandable breath reservoir (20) and pump (30),
and wherein during step (i) of the method the first connector valve (21) is in an open configuration and the second connector valve (22) is in a closed configuration.

16. The method according to claim 14, wherein the pump (30) comprises a filter configured to remove viral particles, or the pump (30) is fluidly connected to a device comprising a filter configured to remove viral particles,
and wherein step (ii) of the method comprises pumping waste breath through the filter configured to remove viral particles before releasing waste breath to the surrounding environment.
